(19) United States
(12) Plant Patent Application Publication
(10) Pub. No.: US 2003/0014788 P1
(43) Pub. Date: Jan. 16, 2003

(54) Apricot tree named 'Mas926'

(76) Inventors: Michael T. Malone, Havelock North (NZ); Jeremy E.B. Davidson, Blenheim (NZ)

Correspondence Address: Michelle Bos, Stratton Ballew PLLC, 213 South 12th Avenue, Yakima, WA, 98902, US

(21) Appl. No.: 09/905,575

(22) Filed: Jul. 12, 2001

(51) Int. Cl. A01H 5/00

(52) U.S. Cl. PLT/186

(57) ABSTRACT

A new and distinct variety of apricot tree (Prunus armeniaca L.) named ‘MAS926’ is described. The new variety is a selection derived from a cross between the apricot varieties ‘Valleygold’ (not patented) and ‘Earliril’ (not patented). The new variety is distinguishable by the dark red blush covering approximately 80% of the fruit surface. The fruit is of medium size with an ovate shape, and has juicy, firm flesh with a sweet tangy flavour. The tree is characterized by vigorous upright growth habit.

### ORIGIN OF THE VARIETY

This invention relates to the discovery and asexual propagation of a new variety of apricot tree. The new and present variety originated from a controlled cross conducted by Michael T. Malone and Jeremy E. B. Davison and was selected by Jeremy E. B. Davison in 1992 from a seedling population planted at the Marlborough Research Centre experimental orchard in Blenheim, New Zealand. Further evaluation was directed by Michael T. Malone. The variety originated from a cross between ‘Valley Gold,’ (seed parent, unpatented) and ‘Earliril’ (pollen parent, unpatented). ‘Valley Gold’ is a Canadian variety derived from Vineland, Canada released as V66052. This selection was named and commercially grown in New Zealand as ‘Valleygold.’

### ASEXUAL REPRODUCTION OF THE VARIETY

The new variety was budded onto ‘Golden Queen’ peach rootstock, the standard apricot rootstock in New Zealand, for the first time in 1992. The resulting trees have propagated true to type showing that the distinctive characteristics are established and transmitted successfully through succeeding generations.

### SUMMARY OF THE VARIETY

The described new variety of apricot tree is a large, vigorous, tall upright tree with an erect tree form. It is a regular and productive bearer of medium sized fruit. The fruit of this new variety is sweet and of high eating quality, and has an attractive dark red blush covering most of the fruit surface.

The new variety differs from the parents by its dark red overcolour covering approximately 80% of the skin surface. The variety differs from the ‘Earliril’ parent by being an upright tree similar to the ‘Valleygold’ variety. The variety differs from the ‘Valleygold’ variety in that ‘MAS926’ has fruit with orange flesh and dark red skin colour.

The variety is most similar to the variety NJA32, marketed as Orangered®, by reason of a high skin colour. The present variety differs from the Orangered® by being of larger fruit size, darker red skin colour and of seven days earlier maturity and upright tree habit.

### BRIEF DESCRIPTION OF THE DRAWINGS

The accompanying photographs show typical specimens of the foliage and the fruit of the new variety. The photograph presented at FIG. 1 was taken shortly after harvest with fruit at eating maturity.

FIG. 1 shows the upper and lower surface of the leaves, and the exterior and sectional view of the fruit divided along the suture plane to show flesh colour, the stone remaining in place; and

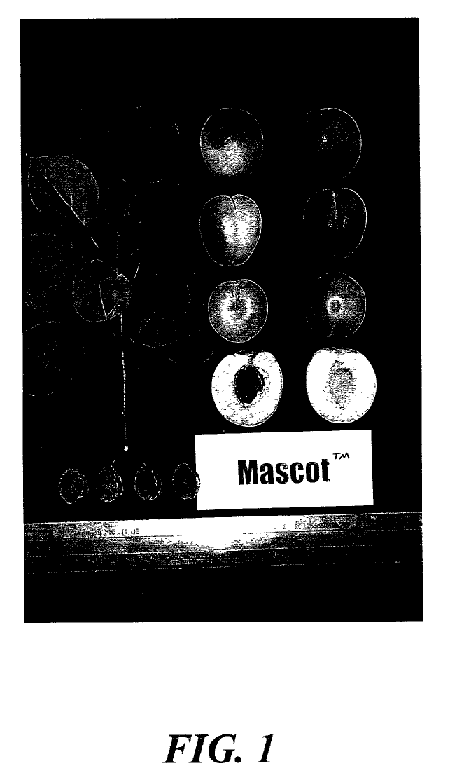

FIG. 2 shows typical fruit and leaves of the new variety.

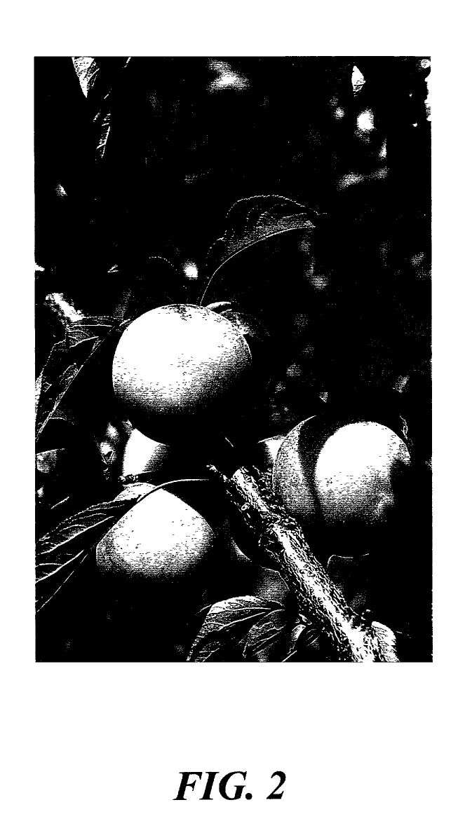

### DETAILED DESCRIPTION OF THE NEW VARIETY

The following is a detailed description of the new variety with colour terminology in accordance with The Royal Horticultural Society Colour Chart 1986 edition. The specimens described were grown in Clyde, Central Otago, New Zealand. The observations were made in the 2001 season on trees that were 7 years old at the time, gown on ‘Golden Queen’ rootstock.

Tree:

Size.—Large.

Vigour.—Vigorous.

Growth.—Habit Tall, erect.

Foliage.—Density Medium.

Production.—Average fruit production.

Trunk:

Size.—Medium.

Texture.—Average roughness.

Colour.—Grey/orange 165A.

Branches:

Texture.—Smooth.

Lenticels.—Average quantity present on branches.

Young shoot—current season growth.—Size Medium Anthocyanin Coloration at Tip Medium.

Leaves:

Average blade length.—91 mm.

Average blade width.—73 mm.

Shape of tip.—Acuminate.

Angle of tip.—Obtuse.

Shape of base.—Truncate.

Incision of margin.—Serrate.

Color—upper surface.—Dark Green 137A.

Color—lower surface.—Brown Green 147B.

Petiole.—Medium length, medium thickness.

Glands.—Medium size, usually 3 or more glands on petiole, generally globose in shape.

Flowers:

Bud size.—Medium.

Bud length.—Medium.

Bud shape.—Round, plump.

Form.—Free.

Flower size.—Medium diameter.

Petal shape.—Broad elliptic shape.

Colour.—White.

Fruit:

Maturity when described.—Eating ripe.

Size.—Medium Average Diameter 48 mm Average Length 52 mm Average Weight 80 g.

Form.—Ovate in suture view.

Suture.—Shallow, extends from stem to apex.

Fruit base.—Rounded, apex is slightly depressed.

Flavour.—Sweet, tangy with good acid balance.

Juice.—Moderate juiciness.

Average sugar content.—180 Brix.

Symmetry.—Along suture predominantly symmetric.

Stem.—Pulls free from stone.

Use.—Dessert, fresh market.

Keeping quality.—Good.

Skin:

Thickness.—Thick skinned.

Texture.—Smooth.

Tendency to crack.—Low.

Pubescence.—None.

Colour.—Orange 26A ground colour with 70% to 80% red 45A blush over colour.

Flesh:

Ripens.—Evenly.

Texture.—Firm, finely grained.

Fibres.—Present, tender.

Flavour.—Sweet/tangy.

Eating quality.—Very good.

Colour.—Orange 26A.

Stone:

Type.—Freestone, slight attachment of flesh fibres along suture.

Average weight.—3.3 g.

Average length.—28 mm.

Average width.—23 mm.

Average stone thickness.—12 mm.

Form.—Ovate.

Base.—Flattened.

Apex.—Rounded curve to apical tip.

Surface.—Smooth, no pits.

Ridges.—Prominent ridge on suture line.

Colour.—Brown 177B.

Tendency to split.—None.

Kernel:

Taste.—Bitter.

Viable.—Yes.

Colour.—Yellow Brown 165B.

Average width.—13 mm.

Average length.—19 mm.

The tree, flowers and fruit may vary in slight detail due to variations in soil types, cultural practices and climatic conditions. The present botanical description is that of the variety grown in Clyde, Central Otago, New Zealand.

We claim:

1. A new and distinct variety of apricot tree named ‘MAS926’ substantially as illustrated and described.
